(19) United States
(12) Patent Application Publication
(10) Pub. No.: US 2023/0160832 A1
(43) Pub. Date: May 25, 2023

(54) BIOLOGICAL SAMPLE QUALITY APPARATUS

(71) Applicant: BSD ROBOTICS PTY LTD, Brendale (AU)

(72) Inventor: Andrew Stewart, Brendale (AU)

(21) Appl. No.: 17/997,152

(22) PCT Filed: May 20, 2021
(86) PCT No.: PCT/AU2021/050474

§ 371 Date: Oct. 26, 2022

(30) Foreign Application Priority Data
May 20, 2020 (AU) 2020901625

(51) Int. Cl. G01N 21/88; G01N 21/01; G01N 33/48; G01N 35/00

(52) U.S. Cl. CPC G01N 21/88; G01N 21/01; G01N 33/48; G01N 35/00732; G01N 2201/10; G01N 2035/00752; G01N 2201/0636; G01N 2201/121; G01N 2201/0693

(57) ABSTRACT

The present invention relates to a biological sample quality apparatus for determining the quality of a biological sample. The apparatus includes a sample receiver for receiving the biological sample. One or more light sources are provided for supplying light to the sample. An image sensor is provided for capturing an image of the lit sample. The apparatus also includes an image processor for image processing the captured image to determine the quality of the sample. Advantageously, image processing may be used to determine the quality of a sample for use in collection sites and screening laboratories so that acceptability can be determined prior to analyzing the sample. Determination that the sample is of sufficient quality (e.g. sufficient biomaterial) prior to analyzing saves wastage of laboratory time and expense of materials and chemicals. The apparatus may be in the form of desktop or hand-held portable variations.

### TECHNICAL FIELD

The present invention generally relates to a biological sample quality apparatus for determining the quality of a biological sample.

In particular, the present invention relates to a method and apparatus for pre-analytical assessment of dried biofluids on filter paper to determine suitability of a biological sample for downstream analysis by chemical, biochemical or molecular test methods. The present invention has particular, although not exclusive application to Dried Blood Spot (DBS) biological samples. Other applicable biological samples include dried saliva, buccal, urine and serum or plasma samples. It is envisaged that the invention will have other applications for biological sample assessment.

The invention can be deployed in several application environments for complete sample monitoring and management. Environments include remote points of sample collection such as birthing centers or hospitals, central analytical and pathology laboratories and at sample materials manufacturing facilities for raw product quality assurance and control. Other environments include neonatal health assessment, personal health profiling and monitoring, and forensic DNA sample collection.

### BACKGROUND

The reference to any prior art in this specification is not, and should not be taken as an acknowledgement or any form of suggestion that the prior art forms part of the common general knowledge.

Dried Blood Spot (DBS) samples are used for sample collection for the purpose of newborn health screening, where drops of blood are placed on a sheet of absorbent filter paper. Once dried the sample can be put in the mail to a central laboratory for processing in a safe and convenient way without the complications of whole blood sample transport. DBS samples are also used for other analytical tests involving blood sample collection for infectious diseases, patient health markers, paternity testing and for drug metabolism studies.

Newborn screening is practiced worldwide to allow early detection of conditions that are either life-threatening or can cause a clinically significant adverse outcome if left untreated. Several analytical methods may be involved in the screening process and the results are subject to the quality of the received sample. DBS quality is normally assessed subjectively by visual inspection in the screening laboratory before analysis. However, the rejection of samples is not standardized, since no specific guidance exists to define the minimum DBS quality acceptance criteria other than a simple visual chart and differences exist between laboratories and individuals for sample acceptability.

The result is a wide variation in practice, with different laboratories accepting or rejecting samples of differing quality, leading to confusion among sample collectors regarding what constitutes an acceptable sample and compromises analytical consistency.

Existing reference materials are published by the Clinical Laboratory Standard Institute which publishes a procedural guide and Standards, including:

NBS01-A6 QG: Specimen Collection and Sample Quality for Newborn Screening Quick Guide

NBS01: Blood Collection on Filter Paper for Newborn Screening Programs, 6th Edition

In both situations, assessment is purely visual and based on either CLSI guidelines and/or local best practice.

Most laboratories processing dried blood spot samples regularly receive insufficient or low-quality samples with significantly increased chance of unsatisfied results, false positives or negatives, increased processing costs, effects on parental and patient stress and increased potential for negative life outcomes.

The preferred embodiment provides for improved quality assessment of a DBS sample for analysis.

Collection of biological samples for clinical and health diagnostics often requires patient sampling at a location remote from an analytical laboratory. Samples are transported to the laboratory and, if in fluid form, the need for handling and safety aspects are complex and costly.

Dried biosamples normally exhibit long-term stability and compact storage without the need for refrigeration. Since the sample is dried the environmental conditions and sample safety are more convenient than fluid sample transport.

Aside from the convenience of sampling and transport, dried samples normally exhibit long-term stability and compact storage without the need for refrigeration. Stability can be improved and sample pre-preparation performed by chemically pre-treating the collection material, for example by adding reagents to lyse cells or stabilise proteins and DNA. The sample collection paper may also be impregnated with indicating dye that changes colour on application of the sample to allow visual location of the sample which may be a clear liquid such as saliva.

DBS Samples for Newborn Health Screening

In the case of blood sampling, normal practice is to puncture the skin with a lancet and apply a large single drop of blood onto the sample media, without touching the area of media with the blood spot. Analytical errors are known to occur if the sample is applied more than once, if the material is scratched, abraded or deformed, all of which affect the absorption of sample per unit area of the sample media and therefore the relative amount of biosample that is later eluted for analysis.

It is essential that the dried sample provides analytical results similar to, or indicative of the original sample, such as whole blood. There are number of confounding factors that influence the quality and subsequently analytical results of dried biological samples, including the size and volume of the sample spot, drying time, age, shape, homogeneity or consistency of the sample across the dried sample area.

Contaminants present in the sample from the sampling process can also affect analytical or DNA test accuracy and examples including glove powder and residues from skin site sterilisation such as isopropyl alcohol.

To ensure that the dried sample provides analytical results similar to or indicative of the original sample, certain conditions must be met and dependent on the application of the sample to the collector and handling of the sample prior to transport.

In the absence of such standards and assessment, analytical results may be unreliable and inaccurately represent the concentration of compounds in a patient sample and can result in misdiagnosis of a critical health condition.

Known factors that can affect analytical results include the size and volume of the sample spot, sample drying time before transport, sample age, shape of the dried spot and consistency of the sample across the spot. Sample consistency is compromised if the sample is not evenly applied or multiple applications are applied to the same area of the collector material. Clearly, if there is insufficient sample to perform a required test, then the analytical result will not represent the amount of a compound in the original patient sample.

Biofluids such as blood are a rich mixture of cells, liquids and compounds such as amino, organic and nucleic acids which provide a means for biochemical and molecular diagnostic testing to determine abnormal health conditions. In the case of biochemical tests, diagnosis is based on a response to a reaction with the native compound in the biofluid and regents or indicator chemicals introduced in precise volumes with the patient sample. The degree of reaction and result depends on the ratios of concentrations of compound in the sample and the introduced reagents. If, therefore, for any reason, the volume or concentration of the patient sample is compromised then so is the analytical result which may lead to an incorrect diagnostic result.

Aside from effects of the sample collection attributed to the physical application, the constituents of cellular and liquid components of blood can also affect the integrity of the sample. Some compounds are found only or substantially within the non-cellular liquid component of blood. Therefore, if the ratio of serum to cellular content, known as haematocrit, is outside normal limits then the analytical result may not accurately reflect the true concentration of a compound in the patient sample.

With dried sample collection of blood, the application of spots to a sample material results in the sample spreading through the fibers of the collection material and dispersing radially from the application point, eventually drying at the edges of the blood spot. In some cases the liquid serum portion and cellular materials can become separated to a degree resulting in ‘serum rings’ around the outer radius of the blood spot, resulting in a non-homogenous distribution of blood components on the sample collector surface (herein referred to as sample consistency).

The dried sample spatial consistency is of high importance for the purpose of analytical preparation, as small disks of the dried spot are removed (sub-sampled by punching the collection material) for analysis, and not normally the entire spot. The location of the sub samples in the dried spot area should ideally not have an influence on the analytical result provided that the sub sample is entirely within the radius of the dried spot and not close to the periphery of the spot. Multiple sub-samples may be taken from one spot to determine one or more health conditions through analytical tests and it is required that there is minimal variation in the amount and content (serum to cell ratio for example) between each sub-sample.

The optical properties of dried blood spots are significantly influenced by the presence of haemoglobin which produced a characteristic red colour with an increased degree of visible light reflection above 590 nm. The ratio of red to green or blue wavelengths is therefore an indicator of haemoglobin, or red blood cell constituents, and is affected by the ageing of the dried sample. Over time dried blood has a brown-coloured appearance rather than red. In this way, by comparison of red and green colour channels of an imaging device, indication can be provided as to the consistency of haematocrit and sample age.

The liquid serum portion of blood exhibits a relatively consistent optical absorption over wavelength and therefore does not show the same characteristic curve as haemoglobin over the visible range of wavelengths. This can be used to effect in determining if an area of sample is serum or contains red blood cells.

If the sample quality is found to be inadequate, then two actions can occur; the analytical laboratory can attempt to remove acceptable areas from the sample medium, or the patient needs to be recalled for a new sample. The former case is practical in terms of timing, though this is a laborious process and samples normally cannot be processed in a high throughput fashion to maintain economies of cost and laboratory efficiency. However, the invention has utility in digitally examining samples and regions within dried blood spots for acceptable zones to remove that are more likely to produce reliable results. This affords time efficiency in avoiding collection of repeat samples.

In the case of a repeat sample collection, delays in obtaining a repeat sample can compromise newborn screening programs where the timing of tests after birth is critical. A further issue is the need to request a patient to take a follow up sample and this is known to result in stress and anxiety, particularly in the case of parents of newborns, and can have prolonged impact, irrespective of physical health condition.

Forensic Sample Collection

Collection of DNA samples using saliva on sample collectors is used for remote and reference sample collection. Saliva can be a direct deposit or may be transferred from a swab or absorbent pad of a collection device.

Dried saliva samples have little colour and when spotted onto white filter paper are not easily visible to determine the presence and location of the sample. for this reason, sample collector materials may contain a coloured dye that changes colour after application of the sample to provide visual indication of the sample application. Saliva samples normally contain DNA within buccal cells that are the target for DNA analysis from an individual for purposes of human identification.

The relevance of the invention to DNA or protein sample collection is to identify the presence, size, conformity and location of a saliva sample. The condition of areas not covered with saliva may also be assessed to determine sample quality statistics and classifiers such as sample age.

In this case the invention includes light illumination at a wavelength complementary to that of the dye colour, in order to best discern the sample image. A typical example is the use of pink indicating dye on sample collectors known as FTA cards (see U.S. Pat. No. 7,748,283) which are used for forensic sample collection and in this case the invention uses a wavelength at green (565 nm) wavelength to provide an image with sufficient contrast between saliva sample and bare areas of the collector.

There is a need for a system to digitally analyse dried biosamples to verify sample quality prior to downstream analysis. It embodies algorithms to determine relevant factors such as sample area and consistency in order to determine parameters and scores that provide human interpretable understanding of the sample quality, including overall quality ratings and pass/fail determination. The invention is described as two embodiments: a benchtop instrument intended for analytical laboratory usage, and a portable unit for use at collection centres and sites.

### SUMMARY OF THE INVENTION

According to one aspect of the present invention, there is provided a biological sample quality apparatus for determining the quality of a biological sample, the apparatus including:

a sample receiver for receiving the biological sample;

one or more light sources for supplying light to the sample;

one or more image sensors for capturing an image of the lit sample; and

an image processor for image processing the captured image to determine the quality of the sample.

Advantageously, image processing may be used to determine the quality of a sample for use in collection sites and screening laboratories so that acceptability can be determined prior to analyzing the sample. Determination that the sample is of sufficient quality (e.g. sufficient biomaterial) prior to analyzing saves wastage of laboratory time and expense of materials and chemicals.

The sample receiver may include a dock for docking a sample card bearing the sample. The dock may include a guide or slot so that a sample card can be inserted into the apparatus in a consistent and repeatable manner. The sample receiver may include a clamping mechanism to temporarily clamp the sample in position. The sample receiver may include a detector (e.g. switch) to detect for the presence of the sample card. The sample receiver may include a moving mechanism to automatically move the sample to and from an image capture position.

The light sources may provide one or more specific wavelengths. The light sources may be adjustable for intensity and/or exposure time. The light sources may include a transilluminator whereby the light emanates from a planar surface (e.g. backlight). The light sources may include a reflector for reflecting light from within a lightbox. The light sources may include a lightbox to direct controlled light toward the sample to provide acceptable image quality free from unwanted reflections and spatial light variation. The light sources may include a light-safe enclosure with minimal internal reflections to ensure integrity and homogeneity of the captured image. The light sources may include one or more LEDs.

The apparatus may include an identifier for identifying the sample. The identifier may read a printed barcode or radio frequency identification tag. The identifier may be fixed to the apparatus.

The image sensor may utilize reflective and/or transmissive scanning of the sample. The image sensor may be a one dimensional (1D) or two dimensional (2D) device. The image sensor may have capability for suitable image resolution and dynamic range to capture the sample image for subsequent image processing and statistical analysis from specific viewing positions relative to the sample. The image sensor may consitute one or more cameras positioned to capture images from one or both sides of the sample using either reflected or transmitted light from the light sources.

The apparatus may include a controller for controlling the apparatus. The controller may control the light source and image sensor to capture digital images. The controller may include the image processor, and may process and store captured images, perform numerical computations, and/or interface with network and data storage devices.

The apparatus may be portable, including an exterior housing to facilitate portability and incorporating a touch screen interface for stand-alone usage. The apparatus may include an internal battery supply for supplying the apparatus in areas where power connection is not available.

The apparatus may include internal or external storage media for storing captured images and data records. The apparatus may include optical filtering to allow specific wavelengths of light to be clearly discerned.

The apparatus may include a communications port for integration to a Laboratory Information System (LIS) or other data system over computer network connections.

According to another aspect of the present invention, there is provided a biological sample quality method for determining the quality of a biological sample, the method including:

receiving the biological sample;

supplying light to the sample;

capturing an image of the illuminated sample; and

image processing the captured image to determine the quality of the sample.

Advantageously, the method may involve spatial computation for downstream removal of one or more acceptable areas of an acceptable sample (e.g. containing consistent biomaterial) for subsequent analysis to provide accurate and repeatable results.

The method step of receiving the sample may involve detecting the presence of the sample and clamping the sample in a fixed position.

The method may involve activating the light.

The method may involve identifying the sample. The step of identifying may involve activating a barcode scanner to automatically record a sample identifier or digitally processing the identifier from the captured image.

The method may involve compensating the captured image prior to display to an operator and/or storage. The compensating may involve spatial and/or intensity correction. The step of capturing may involve optical filtering to allow specific wavelengths of light to be clearly discerned.

The image processing may involve statistical or logistic processing of one or more captured images, from one or more viewing positions of the sample, and at one or more wavelengths of the supplied light to determine the quality of the sample. The step of image processing may involve determining an acceptable dried sample area including location, area and spatial arrangement including boundaries. The step of image processing may involve assessment of sample density and consistency across the sample. The image processing may involve classifying the sample from statistical data. The statistical data may relate to blood hematocrit and age of sample estimates based on wavelength comparisons.

The image processing may involve computation of human-relatable metrics allowing direct comparison of human visualization with machine image processing results. The image processing may involve a final pass or fail result. The image processing may involve determining an overall quality factor by assessing image processing values against parametric constants which are able to be adjusted by an operator.

The image processing may involve computation of acceptable sampling regions of the sample. The regions may be determined using parameters relating to the circularity of sample spot; consistency of image values within each sample spot; texture analysis of the sample spot or image area comparisons based on wavelengths or ratios of wavelengths. The regions may also be determined from user-specified values relating to specific laboratory workflow, such as the number and size of sub-samples to be removed from the sample.

The image processing may involve determining the centroid coordinates of suitable circular, of a specified diameter (e.g. 3.2mm circular punch sites), areas containing sample areas to be removed. The centroid coordinates may be referenced to a datum point on a sample card so that the card can be inserted to other equipment for automatic removal (e.g. punching) of the areas to be removed.

The method may involve storing a data record including the captured image, computed information including statistics and sample identification details.

The method may compute, display and store classifications of each blood spot and for the entire sample. The user may have the facility to override classifications if the user disagrees with a displayed classification, allowing subsequent improvement of classification algorithms based on user feedback.

The method may involve displaying and recording results of sample quality determination.

Embodiments may acquire and digitally scan sample images, display results to the user and store records of results for subsequent transfer to external information systems. The apparatus is intended, in two main embodiments, for use in high-throughput laboratories as a benchtop instrument and as a compact portable unit for use by sample collection personnel, herein referred to as ‘desktop’ and ‘portable’ embodiments.

Usage of the apparatus may involve inserting a dried sample collector, typically comprising a section of filter paper sandwiched in a cardboard frame (herein referred to as a sample collection or collector card) carrying one or more spots of dried biofluid such as blood or saliva. With the card in position the apparatus may activate a light source and capture one or more images which are processed to generate statistics and quality metrics. Since time is of the essence in high-throughput scenarios, the image illumination and acquisition time is preferably minimal and with ease of sample presentation and positioning.

Embodiments of the invention include the arrangement of the apparatus in various embodiments, methods of analysis and means of quality assessment. Details of the embodiments include the sample presentation arrangement, controlled light sources, imaging method, system elements and functions and user interface.

Light may be applied to the sample as described in Patent US 2013/0043374, whereby a light source illuminates the sample material and an image is acquired from the opposing side of the material, i.e. in transmissive mode. In this embodiment, light may also be applied from the same side as the imaging device, that is, in reflective mode. Either one or two images of the sample may be obtained using these methods and used independently or in combination as input to image processing algorithms. Advantageously, collection of images from both sides of the sample card may enable determination of whether the liquid sample has homogenously saturated the entire spot area or has dried on one surface only in some areas.

Imaging may take a 1 or 2-dimensional format. In the case of 1 dimensional a linear array, linear lens and light strip may be used to scan the sample, line by line. A 2D format preferably involves an area image sensor. The 1D format affords compact optics and is suited to a compact and portable embodiment of the invention. For a desktop instrument, a 2D sensor offers fast image capture which is of prime importance for usage in a high throughput laboratory.

In the case of a 1D format, the light source can be arranged adjacent to the linear detector array and this is common for Contact Image Sensors that are used for document scanning applications. Where a transmissive light is required, an LED backlight, comprising a thin sheet of light scattering material and illuminated at the edges by LEDs provides a compact light source able to consistently illuminate an area. This implementation is also amenable to lightweight and lower power applications for a portable embodiment of the invention.

The apparatus may provide quantification of the number of sub samples meeting minimum quality criteria that can be removed from the sample.

The 1D format may require a mechanical means to move the linear image sensor and lens array over the surface area of the sample that is required to be checked. The movement may be controlled such that the position of the image sensor in the axis of movement is precisely known since each line scan represents a spatial position of the sample. The line scans may also be taken at appropriate physical intervals to ensure the spatial consistency of the image in the direction of movement. This may be achieved using a stepper motor driven mechanism and position switch or using a motor-driven mechanism with position feedback such as an encoder or potentiometer.

For both 1D and 2D embodiments, it is desirable to fix the position of the sample during the imaging process and to commence imaging only when the sample is in an appropriate position to enable full view of the dried biosample areas. This may be achieved using a clamping mechanism that activates once the sample is in position. Said clamping mechanism may be electronically activated, in the case of the benchtop instrument, or by mechanical means such as the closure of a latch after inserting the sample card in the portable embodiment.

Detection of the card in position may be signaled by a miniature mechanical (detector) switch, an optoelectronic sensor, or by the imaging system operating continuously to detect the sample in an appropriate position and alignment. To facilitate placement the invention may incorporate mechanical guides to guide the sample positioning along one or more edges of the sample material.

In one embodiment, the use of narrow wavelength LEDs and optical bandpass filters for illumination allows more discrimination of samples image at different wavelengths to determine the hemoglobin light absorption and response for determination of sample hematocrit consistency and indication of blood spot age.

In the case of samples with indicating dye, a narrow wavelength is not critical although may assist with determination of collector quality, in that to determine quality of the sample collector in areas outside of the sample spot because the sample collector can be compromised by humidity or age in which case the intensity of the dye fades. Multiple wavelengths may be therefore used to compare red and green intensities as a quality factor.

Optionally, the use of LED illumination at one or more colours also permits the use of a monochromatic image sensor, with LED colours switched to capture the image at desired light wavelengths. Alternately, white or multicolor LEDs may be used in conjunction with a colour image sensor to simultaneously acquire images at multiple wavelengths. A further alternative may be to use an optical grating and area image sensor to acquire line scans at multiple wavelengths.

Sample collector materials may contain a coloured dye that changes colour after application of the sample to provide visual indication of the sample application. In this case, the embodiment includes light illumination at a wavelength complementary to that of the dye colour, in order to best discern the sample image. A typical example is the use of pink indicating dye on sample collectors known as FTA cards (see patent U.S. Pat. No. 7,748,283) which are used for forensic sample collection and in this case the invention uses an array of green LEDs, Kingbright APTD3216MGC with emission centered at 570 nm wavelength to provide an image with sufficient contrast between sample and bare areas of the collector.

The results produced by the embodiment are provided for each sample which is associated with a specific individual. Hence it is preferred to have the facility to link the output results from the apparatus, with the sample identifier. For this purpose, sample collection devices are typically printed with a barcode for machine recording of an identification number. The presence of such a barcode on the sample allows the apparatus to electronically read, display and store the identification number for each sample and to store individual record files including the sample identification number.

In the embodiment of a portable apparatus, plug-in chargeable internal batteries provide system power, with internal components requiring low voltage DC power, and an on/off switch or preferentially, a button activated standby circuit.

Results computed by the invention may relate to known parameters of dried sample quality and include sample spot diameter, shape, colour and image intensity consistency. Optionally, record files are created for each sample and saved with the sample identifier (barcode number) and time stamp.

Embodiments of the invention include a control system that includes a microprocessor, volatile and non-volatile memories, digital interfaces, and clock circuits. System firmware may include peripheral device interfaces for memory card, communications ports (USB), internal optical components (controlled light source and image sensor) and a graphical user interface. in the case of the portable embodiment, electronic functions include a contact image sensor and motor driver interface for the transport mechanism, and a rechargeable internal battery pack and power management circuitry. Optionally, the system includes wired or wireless communication interfaces (Wifi) to interface with external information systems.

The user interface may comprise an illuminated graphical LCD and touch screen interface with graphics and numeric displays arranged over multiple virtual screens to display overview information and more detailed results.

The graphical interface may also provide for adjustment and selection of basic user settings. Through a dedicated communications port (USB), more advanced settings and calibration data and functions may be accessed, and internal records downloaded.

Alternately, embodiments of the invention could be controlled and operated as a peripheral device (USB or wireless) to a computer with software performing functions of graphical interface, record storage and routing operation.

Further applications of the invention are in quality control assessment for routine manufacture of sample collectors and materials, for post-diagnosis of samples that return abnormal or unexpected analytical results, and as a training tool for collection personnel where quality metrics provide quantitative feedback.

Any of the features described herein can be combined in any combination with any one or more of the other features described herein within the scope of the invention.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will make reference to a number of drawings as follows:

FIG. 1 is a front view of a sample quality apparatus in accordance with a desktop embodiment of the present invention;

FIG. 2 shows a sample card including newborn DBS samples for processing by the apparatus of FIG. 1;

FIG. 3 is a block diagram of the apparatus of FIG. 1;

FIG. 4 is a flowchart of a DBS sample quality method performed using the apparatus of FIG. 1;

FIG. 5 is an isometric view of a sample quality apparatus in accordance with a desktop embodiment of the present invention;

FIG. 6 is an orthographic drawing of the sample quality apparatus of FIG. 5;

FIG. 7 is an exploded view of the sample quality apparatus of FIG. 5;

FIG. 8 is a sectional view of the sample quality apparatus of FIG. 5;

FIG. 9 is a partial view of the sample quality apparatus of FIG. 5 showing a sample card clamping mechanism from the inside of the apparatus;

FIG. 10 is a partial front view of the sample quality apparatus of FIG. 5.

FIG. 11 is an isometric view of a sample quality apparatus being loaded with a sample card in accordance with a portable embodiment of the present invention;

FIG. 12 is an exploded view of the portable sample quality apparatus of FIG. 11;

FIG. 13 is an orthographic drawing of the sample quality apparatus of FIG. 12;

FIG. 14 is a block diagram of the portable sample quality apparatus of FIG. 12;

FIG. 15 shows various options for arranging imaging components for the portable sample quality apparatus of FIG. 12;

FIG. 16 is a schematic drawing of 2D image sensor circuity of the portable sample quality apparatus of FIG. 12;

FIG. 17 is a schematic drawing of 1D image sensor circuity of the portable sample quality apparatus of FIG. 12;

FIG. 18 is a schematic representation of controlled light source circuitry;

FIG. 19 shows characteristic optical spectral transmission of dried blood and sample card areas;

FIG. 20 shows an example of a DNA collector device with sample collection card comprising indicating paper;

FIG. 21 shows characteristic optical spectral density (light transmission) for saliva on pink indicating paper (Copan Nucleic-Card);

FIG. 22 shows a user interface Home screen;

FIG. 23 shows a statistics screen of the user interface;

FIG. 24 shows a classifications screen of the user interface; and

FIG. 25 shows a settings screen of the user interface.

### DETAILED DESCRIPTION OF PREFERRED EMBODIMENTS

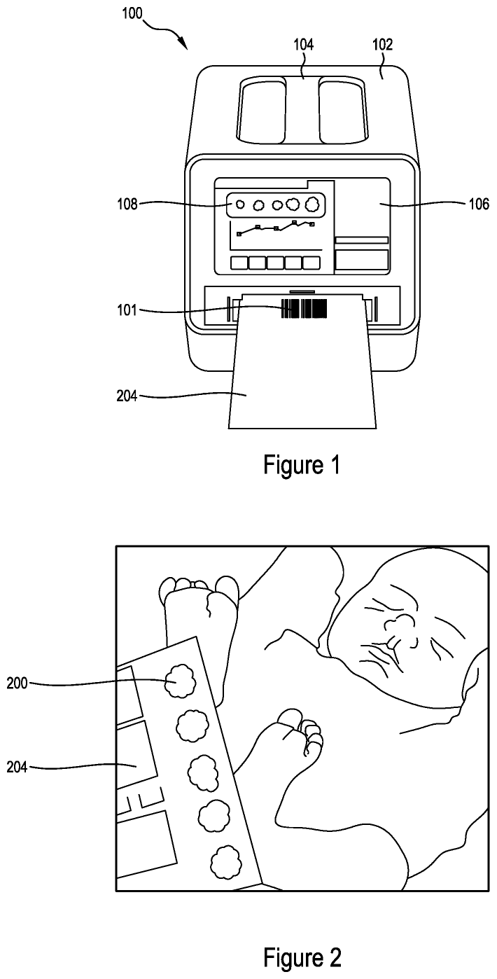

According to an embodiment of the present invention, there is provided a biological sample quality apparatus 100 shown in FIG. 1. The apparatus 100 is suitable for determining the quality of a DBS (i.e. biological) sample 200 shown in FIG. 2. As can best be seen in FIG. 2, the sample 200 is taken from a newborn 202 and applied to a sample card 204.

Returning to FIG. 1, the apparatus 100 includes a dock 101 (i.e. receiver) for docking the sample card 204 bearing the sample 200. The apparatus 100 is portable and includes an exterior housing 102 with a handle 104 to facilitate portability and incorporates a touch screen 106 for stand-alone usage. The touch screen 106 can display a compensated captured image 108 of the sample 200 and may display optimal punch site locations overlaid on the sample image. Digitally processed versions of the image, such as the used of false-colour to highlight acceptable and unacceptable regions of the sample, may be displayed to provide in-depth visual indication of the sample quality.

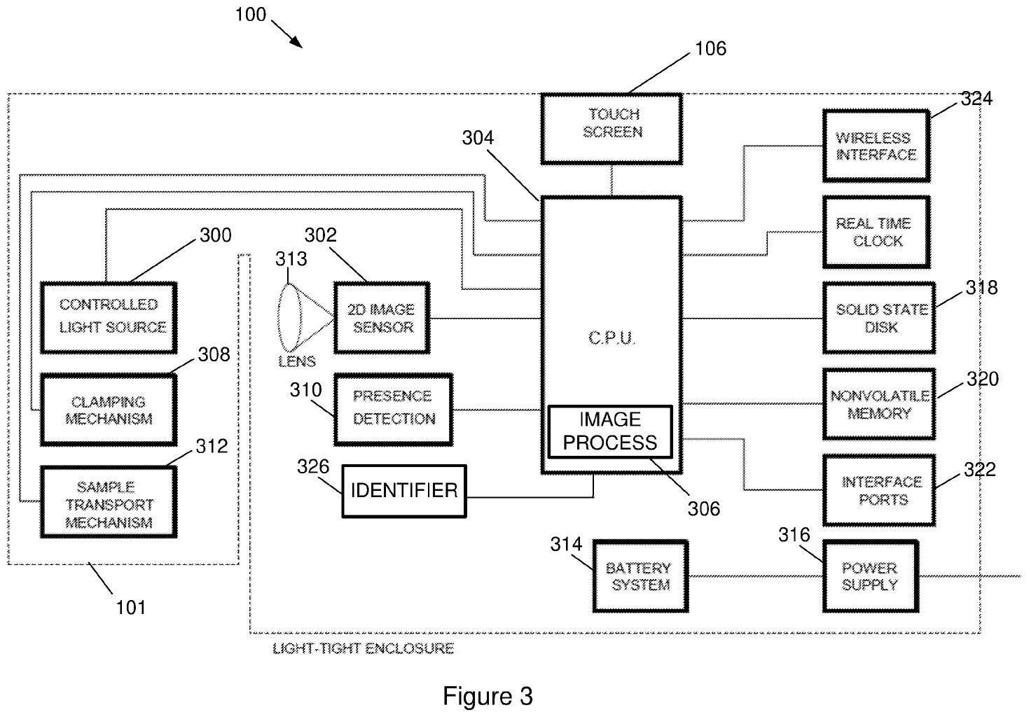

Turning to FIG. 3, the apparatus 100 includes one or more controlled light sources 300 for supplying light to the sample 200. An image sensor 302 is provided for capturing an image 108 of the lit sample 200.

The apparatus 100 further includes a controller 304 for controlling the various functions of the apparatus 100. The controller 304 includes a digital image processor 306. The image processor 306 image processes the captured image 108 to determine the quality of the sample 200.

Advantageously, image processing is used to determine the quality of the sample 200 for use in collection sites and screening laboratories so that acceptability can be determined prior to analyzing the sample. Determination that the sample 200 is of sufficient quality (e.g. sufficient biomaterial) prior to analyzing saves wastage of laboratory time and expense of materials and chemicals. The apparatus 100 is described in detail below.

The dock 101 includes a guide or slot so that the sample card 204 can be inserted into the apparatus 100 in a consistent and repeatable manner. Further, the dock 101 includes a clamping mechanism 308 to temporarily clamp the sample card 204, and therefore the sample 200, in position.

The apparatus 100 includes a presence detector 310 (e.g. switch) to detect for the presence of the sample card 204. The dock 101 includes a sample transport mechanism 312 to automatically move the sample 200 to and from an image capture position.

The light sources 300 include one or more LEDs providing one or more wavelengths. The light sources 300 are adjustable for intensity and exposure time. The light sources 300 can include a transilluminator whereby the light emanates from a planar surface (e.g. backlight) a reflector for reflecting light from within a lightbox or a lightbox to direct controlled light toward the sample 200 to provide acceptable image quality free from unwanted reflections and spatial light variation. The light sources 300 include a light-safe enclosure with minimal internal reflections to ensure integrity of the captured image 108.

The image sensor 302 has capability for suitable image resolution and dynamic range to capture the sample image 108 for subsequent image processing and statistical analysis from specific viewing positions relative to the sample 200. The image sensor 302 includes one or more cameras positioned to capture images 108 from one or both sides of the sample 200 using either reflected or transmitted light from the light sources 300. The apparatus 100 also includes optical filtering 313 to allow specific wavelengths of light to be clearly discerned.

The apparatus 100 includes an internal battery supply 314 for supplying the apparatus 100 in areas where external power connection 316 is not available. Further, the apparatus 100 includes internal storage media, in the form of solid-state disk 318 and nonvolatile memory 320, for storing captured images 108, statistics, classifications, identification code and quality records relating to each sample

Further, the apparatus 100 includes a communications port 322 and wireless interface 324 for integration to a Laboratory Information System (LIS) or other data system over computer network connections. In this manner, records and images can be stored on external storage media.

The apparatus 100 also includes a fixed identifier 326 for identifying the sample 200. In particular, the identifier 326 reads a printed barcode or radio frequency identification tag on the sample card 204.

The controller 304 controls the apparatus 100, and in particular the light source 300 and image sensor 302 to capture digital images 108 of the sample 200. Further, the controller 304 processes and stores the captured images 108, performs numerical computations, creates records of quality assessment and interfaces with network 322 and data storage devices 318, 320.

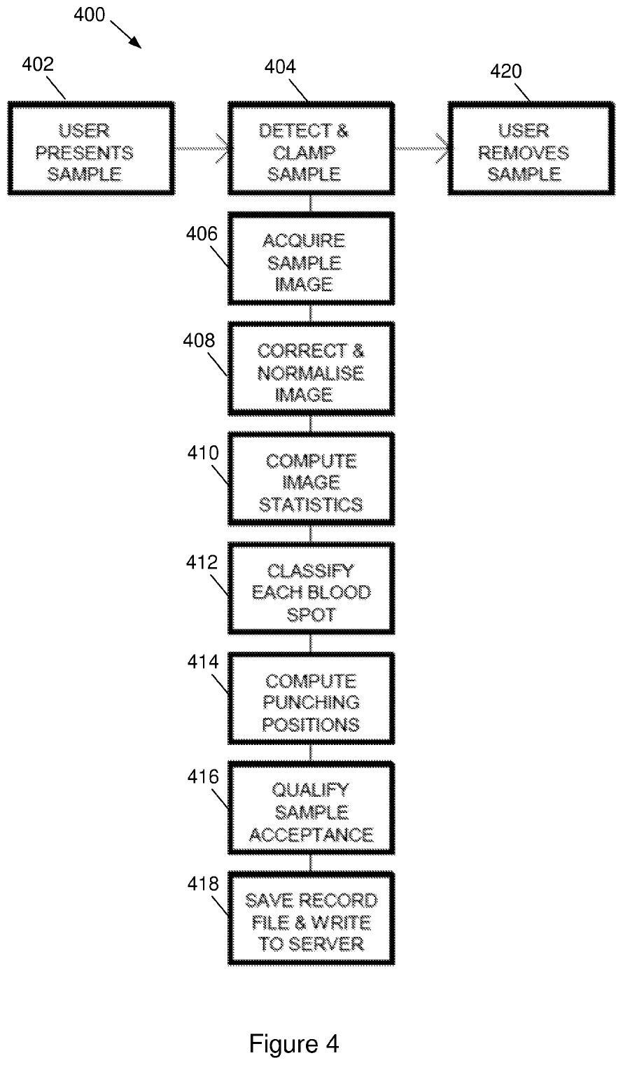

A method 400 for determining the quality of the DBS sample 200 using the apparatus 100 is now described below with reference to FIG. 4.

At step 402, the dock 101 receives the card 204 bearing the sample 200 from an operator.

At step 404, the presence detector 310 detects the presence of the sample card 204 and the clamping mechanism 308 clamps the sample 200 in a fixed position. The light source 300 is activated to supply light to the sample 200.

At step 406, the image sensor 302 captures the image 108 of the lit sample 200. Optical filtering 313 is provided to allow specific wavelengths of light to be clearly discerned.

At step 408, image processor 306 compensates the captured image 108 prior to display to the operator on the screen 106 and storage in storage devices 318, 320. The compensating involves spatial and intensity correction, and normalisation.

At step 410, image processor 306 performs statistical and logistic processing on one or more of the captured images 108, from one or more viewing positions of the sample 200, and at one or more wavelengths of the supplied light when determining the quality of the sample 200. The image processing involves determining an acceptable dried sample area including location, area and spatial arrangement including boundaries. The image processing further involves assessment of sample density and consistency across the sample 200.

At step 412, image processor 306 classifies the sample 200 from the statistical data. The statistical data relates to blood hematocrit and age of sample estimates based on wavelength comparisons.

At step 414, image processor 306 determines regions of the sample 200 for removal. The regions are determined using parameters relating to the circularity of sample spot; consistency of image values within each sample spot; texture analysis of the sample spot or image area comparisons based on wavelengths or ratios of wavelengths. The image processing involves determining the centroid coordinates of suitable circular, of a specified diameter (e.g. 3.2mm circular punch sites), areas containing sample areas to be removed. The centroid coordinates are referenced to a datum point on the sample card 204 so that the card 204 can be inserted to other equipment for automatic removal (e.g. punching) of the areas to be removed.

At step 416, the image processor 306 computes human-relatable metrics, such as CLSI criteria, allowing direct comparison of human visualization with machine image processing results. The image processing involves determining an overall quality factor by assessing image processing values against parametric constants which are able to be adjusted by the operator. The image processing also provides a final pass or fail result, as determination of the quality of the sample 200. The operator then has opportunity to review summary or detailed statistics and to override any classification for storage in the quality record.

At step 418, the apparatus 100 stores a data record including the captured image 108, computed information including statistics, sample classifications and sample identification details from the identifier 326. The identifier 326 identifies the sample. 200 by activating a barcode scanner to automatically record the sample identifier or digitally processing the identifier from the captured image 108.

At step 420, the operator removes the sample 200 (e.g. containing consistent biomaterial) from the apparatus, steps 402, 404, 406 and 420 taking the minimum time to capture the sample image or images. This sample can thus be removed and handled prior to completion of image processing and recording to achieve a high number of samples to be tested in the shortest possible time

The foregoing method 400 can be used to supplement or eliminate subjective human assessment. It provides benefits enabled by the adoption of digital best practice for health laboratory operations. The value presented to the end customer is to reduce excessive time to analysis which can be critical for newborn health, improve reliability of test results, save on laboratory and transport wastage and to avoid human stress in obtaining new samples.

The foregoing method 400 indirectly saves time of the professional staff as well as reduce the stress arises when an ‘insufficient’ sample are collected, requiring concerned parents to bring their baby back in for recollection. As the apparatus 100 is portable it can be used at convenient locations such as hospital wards and collection rooms.

A further benefit can be realized through digital assessment for training and performance monitoring of sample collection personnel for sustained quality improvement in collection practices.

The apparatus 100 provides a user interface to allow collection or entry of identification information for identification and association of record data for the patient demographics, sample identification (barcode), site name or map location, user name (collection person) and instrument identification (serial number).

Sample quality records created by the apparatus 100 can be used for reports categorized by collection location, collection person, and region. Patient demographics entered at the user interface 106 or by other means can be used to link patient records and physical samples through a laboratory information management system and patient records databases.

Through connection to an electronic network such as Wi-Fi or ethernet 324, the records, comprising statistics, results of sample classification and sample images, can be sent to the central laboratory for compilation of reports and alerts which may be used for short term quality rectification or longer term quality improvement programs and warnings of sample problems. Such a system serves to signal a need for an immediate sample recollection and prevent the need for sample recollection after the time and cost expense of sending to the central laboratory for assessment by laboratory staff.

Key to the apparatus 100 is the determination of sites on the dried sample 200 to remove, normally using a paper punch, for the optimum or acceptable sites to ensure best analytical results. Part of the quality and acceptability criteria is therefore optical assessment to determine if there is enough area of acceptable quality blood present on the collected sample 200. The parameters for acceptability are determined and programmed by central laboratory or laboratory management staff. Prospective punch positions are computed and included in the record data.

Ultimately the apparatus 100 provides consistent sample quality and quantity by optically analyzing biological fluids absorbed on a supporting material such as filter paper to determine the most suitable spatial locations for removing sections from the supporting material containing an adequate quantity of sample constituents, including small molecules such as amino acids, larger molecules such as nucleic acids, lipids enzymes and proteins, and cells to assist with accuracy and repeatability of subsequent analytical assays.

Information from the apparatus 100 can be sent to the central laboratory from multiple sites and managed on a server with software to access, manage the data and interface with the laboratory information management systems. The apparatus 100 can also facilitate patient demographic entry at the collection point together with sample quality records and sample images for transmission, storage, and assessment at central laboratories.

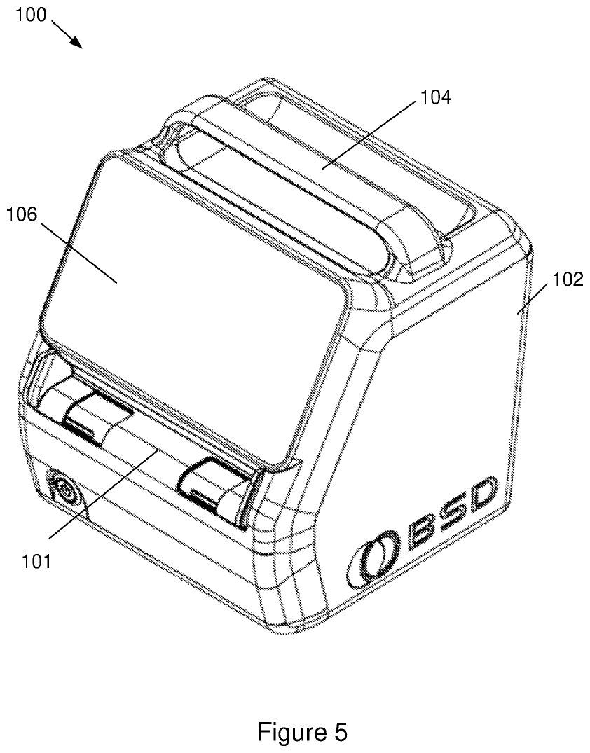

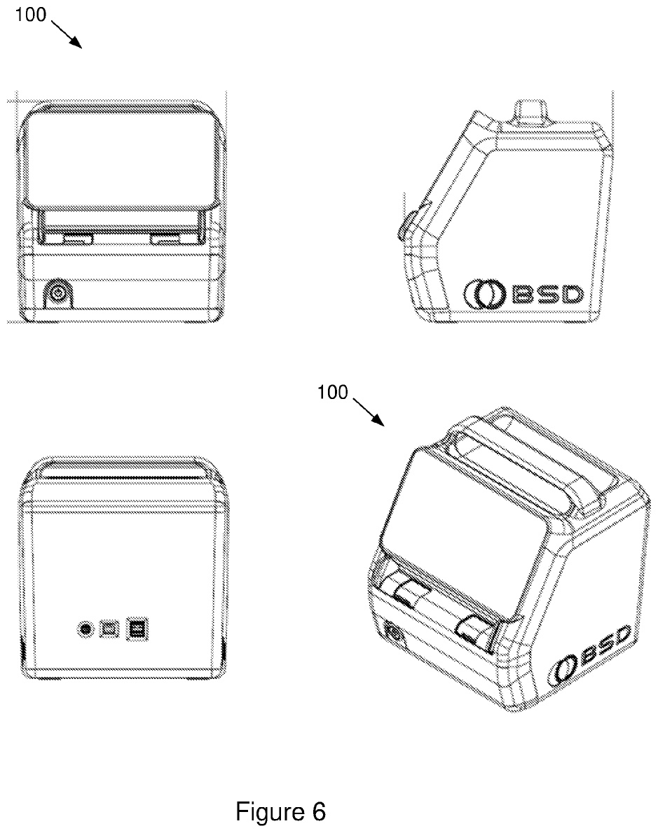

FIGS. 5 and 6 show a desktop embodiment of the apparatus 100. Like reference numerals refer to like features previously described.

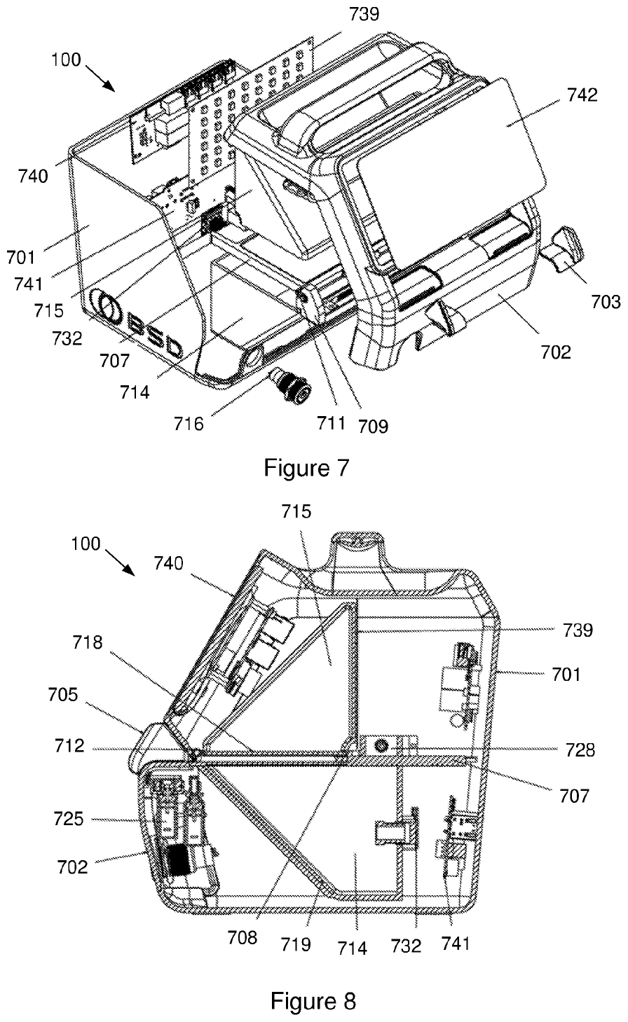

FIGS. 7 and 8 shows features of the apparatus 100 as indicted in the table below.

| | |
|---|---|
| 701. Rear housing | 732. 2D image sensor and reflective |
| | illumination LEDs |
| 702. front housing | 714. Mirror mount |
| 703. sample card guides | 716. Power button |
| 707. card platform | 711. Card clamp beam |
| 709. card clamp mechanism | 742. Graphic LCD and touch screen |
| support | with single board computer |
| 740. controlled light source | 739. Controlled light source LED board. |
| circuit board | |
| 741. Interface circuit board | 718. upper glass support for card |
| | platform |
| 715. light box | 705. sample card entry assembly |
| 712. Card clamp mechanism | 725. Card guides positioner |
| 708. Sample card rear stop bar | 719. Mirror |
| 728. sample card ejector motor | |

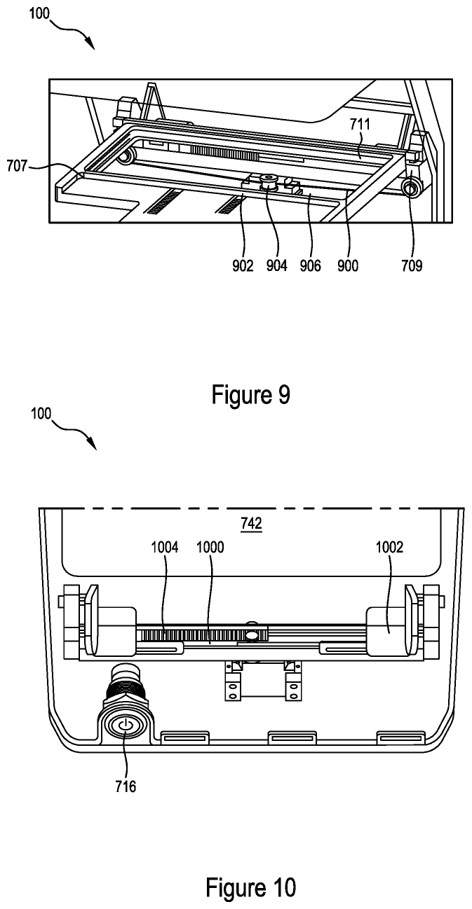

FIG. 9 shows the sample card clamping mechanism from the inside of the apparatus 100. The foreground shows the card platform 707 and gear racks for the card clamp mechanism 709. Springs (not shown) act against the cord tension.

A recess 900 in the platform 707 provides for lower cover glass. Looking through the glass can see the motor 902, spindle 904 and cord 906 underneath that is used to pull in or let out the card guides 725.

The supports 709 are shown for the card clamp bar 711, which is a cylindrical rod across the front of the instrument that clamps down on the sample card 204 when in position.

FIG. 10 shows the LCD panel 742. A card entry slot 1000 is provided with card guides 1002 on each side. A rack and pinion 1004 and motor drive the position of the card guides 1002. The card guides 1002 can also be adjusted by hand in an alternative embodiment.

The on/off button 716 hides the card guide positioning motor 725.

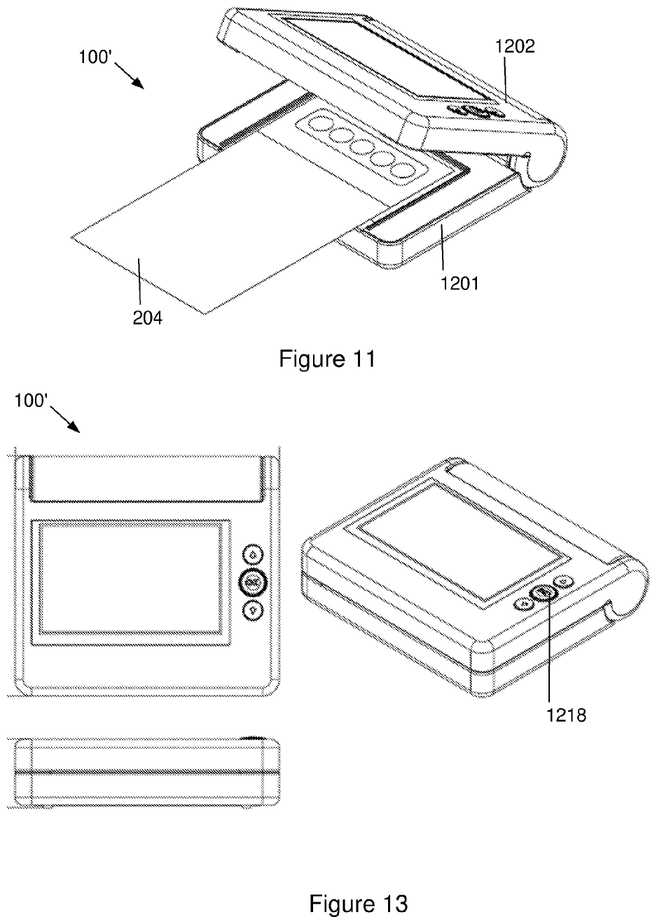

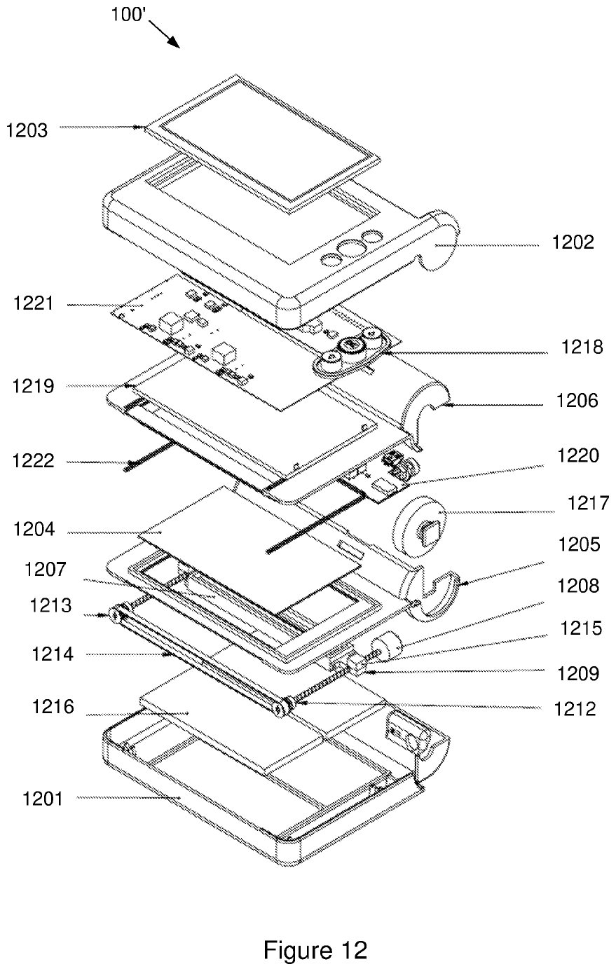

FIG. 11 shows a portable apparatus 100′ being loaded with a sample card 204, with an exploded view shown in FIG. 12.

The card 204 is inserted and supported by a glass panel 1204 under the card 204. Under that sheet of glass 1204, is the contact image sensor 1207 and transport mechanism. The housings 1201, 1202 are hinged at the rear to allow the card 204 to be inserted and then closed for scanning and checking.

A legend for the portable apparatus 100′ shown in FIG. 12 is as follows:

1201—bottom housing—plastic ABS or similar

1202—upper housing—plastic ABS or similar

1203—LCD Touch screen panel with backlighting—typically 5″ colour graphic LCD with capacitive or resistive touch interface

1204—Lower support glass—sample card sits on the glass and provides a window for the contact image sensor to see the bottom of the sample card 204

1205—Card platform lower support—retains the lower glass, ABS

1206—Card platform upper support—retains upper glass and provides some guides at side of frame to slide card in, ABS.

1207—Contact Image Sensor module—moves in a ‘Y’ axis plane—back to front. Contains an image sensor, LEDs and lens array.

1208—Drive motor—servo DC or stepper motor. Drives the worm gear at one side.

1209—Locking magnet (moves to a position to retain the two housings 1201, 1202 and clamp the sample card 204.

1212—Drive nut—moves along the worm (rod with thread) as it turns.

1213—Idler pulley—is driven from the drive side to ensure balanced movement from both sides of the contact image sensor 1207.

1214—Drive belt—couples the drive and idler worm drives.

1215—Contact Image Sensor drive coupling—mechanically connects the contact image sensor to the worm gear nut.

1216—Battery Packs—rechargeable lithium or NiCad thin format cells.

1217—Hinge Pin assembly—fits on both sides to form the hinge.

1218—Sealed Keypad—three button keymat shown in FIG. 13 with resistive contacts to the processor PCB.

1219—Backlight module—with multi-colour controlled light source LEDs to illuminate the sample card 204 through the top support glass.

1220—Interface PCB—includes power and USB connectors and interface components.

1221—Processor PCB—contains digital system components including microcontroller, memory, display interface and peripheral items eg real time clock.

1222—Flexible seal—retains sample card and blocks light entry when enclosure is in the closed position.

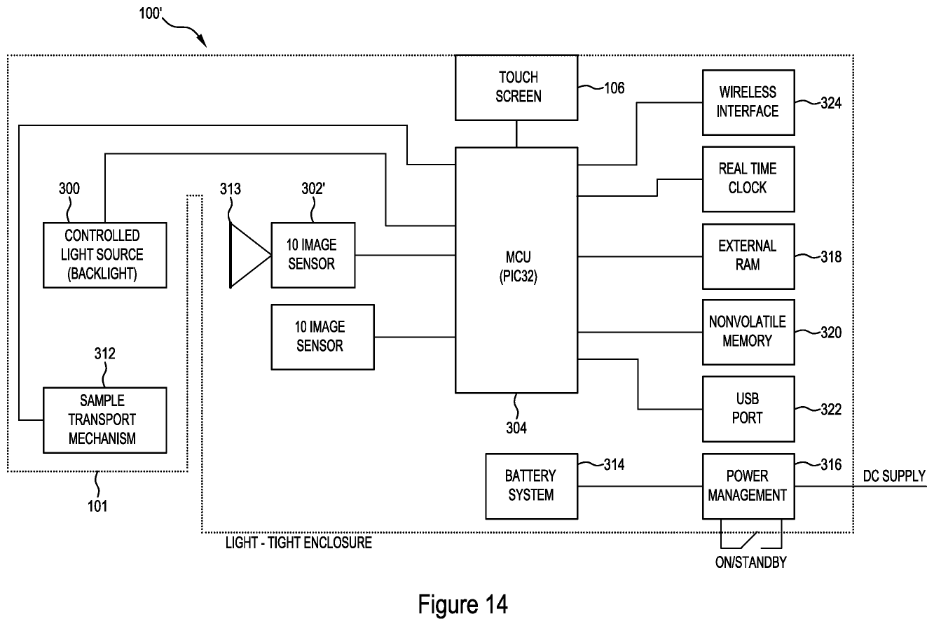

FIG. 14 shows a block diagram of the apparatus 100′, where like reference numerals refer to like features previously described.

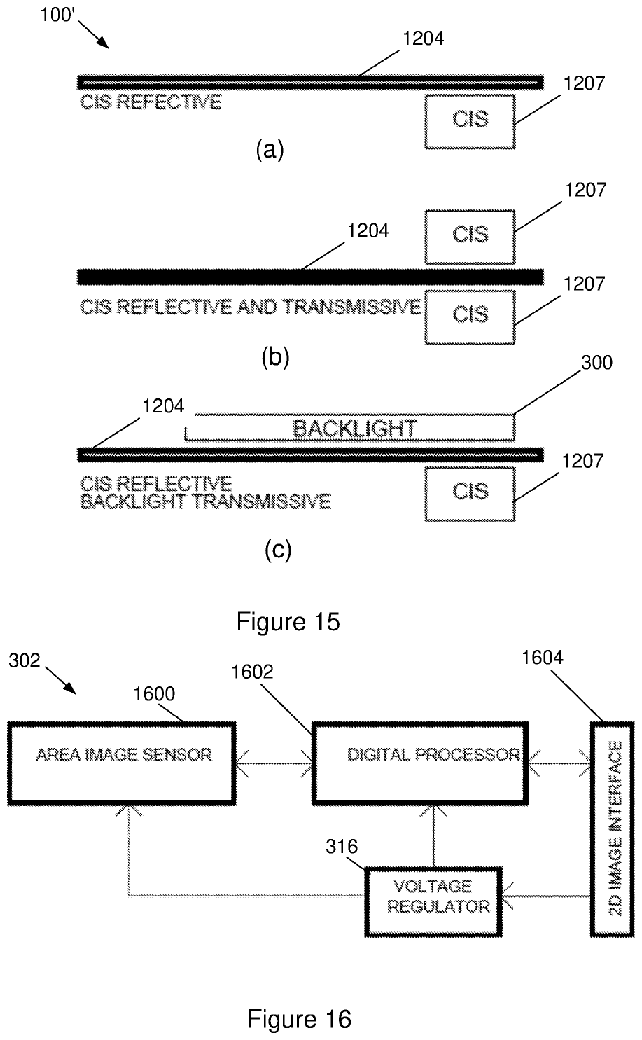

FIG. 15 shows options for arrangement of imaging components for the portable apparatus 100′ (all images are right to left movement of scan).

The image components of FIG. 15a are arranged so that the contact image sensor (CIS) 1207 scans the bottom of sample card 204.

The image components of FIG. 15b are arranged so that the contact image sensor (CIS) 1207 can scan reflective top, reflective bottom, and transmissive through the card 204. LEDs in each CIS 1207 are able to be turned on or off.

The image components of FIG. 15c are arranged so that one CIS 1207 and a backlight 300 are provided over the area of the sample card 204. The processor 304 can turn on the backlight 300 and turn off LEDS in the CIS 1207 to get a transmissive (backlit) image. Alternatively, the processor 304 can use the CIS LEDs only to get a reflective scan.

FIG. 16 shows the 2D image sensor 302 having an Area Image Sensor 1600, which is a module to capture light exposure over a number of pixels (e.g. MT9J001) and having a digital interface 1604. A digital processor 1602 communicates with the image sensor 1600 and provides a suitable interface format to the CPU 304. In one example this may be via USB interface. The digital processor 1602 and image sensor 1600 operate at stable voltages, provided by a voltage regulator 316.

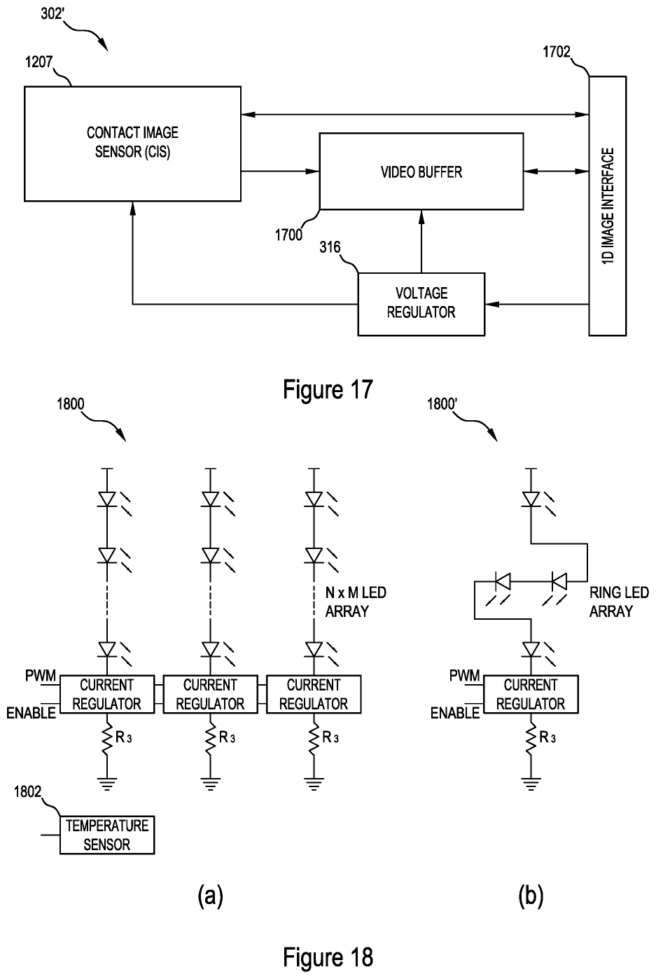

FIG. 17 shows the 1D image sensor 302′ for portable apparatus 100′. The CIS 1207 includes a line scanning sensor, a typical example being TAOS TSL1410R. The output is an analog signal that is clocked out for each pixel in an 1×N array. A video buffer 1700 accepts the analog output and provides a low impedance output to the image interface 1702, which may be an analog input to a microcontroller 304. The interface 1702 also includes digital logic signals to perform the clocking function required by the CIS 1207. The interface 1702 further includes controlled current (as shown in FIG. 18) to drive illumination LEDs within the CIS 1207. The video buffer 1700 and CIS 1207 operate with a stable voltage source, as supplied by the voltage regulator 316.

FIG. 18 shows the controlled light source circuity 1800 driven by the CIS 1207.

FIG. 18a shows a sample of the arrangement for the desktop controlled light source PCB. There are 24 LEDs in total. A temperature sensor 1802 on the PCB allows for thermal management of the LED temperatures.

FIG. 18a shows the circuity 1800′ used for both the portable backlight 300 and within the Contact Image Sensor 1207. The circuitry 300′ also applies to the LED ring that sits around the image sensor lens for the desktop apparatus 100. This allows a reflective image to be taken by illuminating the underside of the card 204 via the mirror.

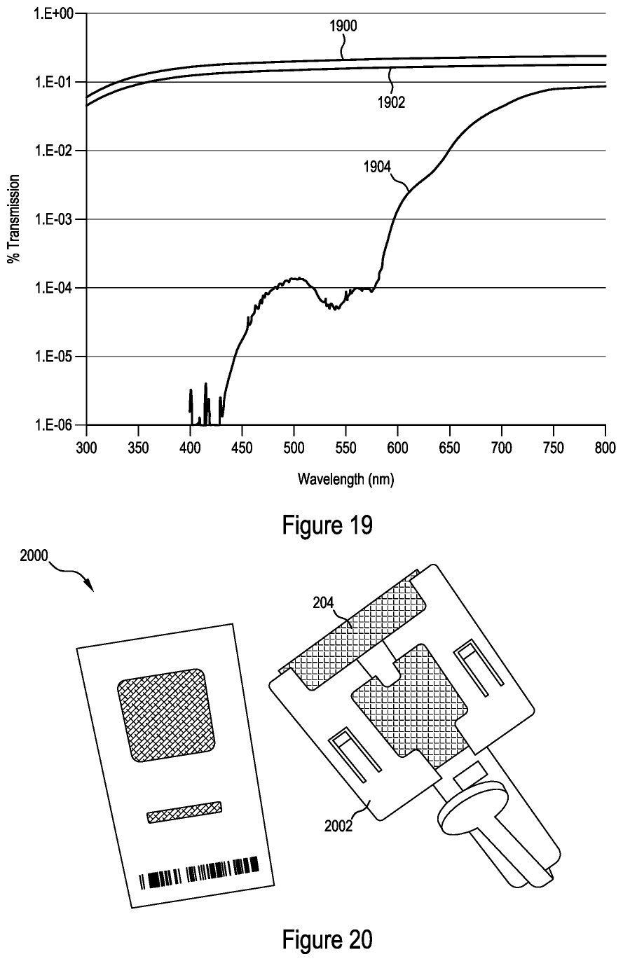

FIG. 19 depicts characteristic optical spectral transmission of dried blood and sample card areas. The blue line 1900 shows the spectral response of filter paper, red line 1902 shows the spectral response of the ink printed areas on the filter paper; and green line 1904 shows the spectral response of dried blood on filter paper. This graph guides the choice of wavelengths to see the contrast of haemoglobin on the sample card 204.

FIG. 20 shows an example of a DNA collector device 2000 with sample collection card 204 comprising indicating paper. The sample card 204 is removed from the holder 2002 prior to use with the apparatus 100 and for subsequent analysis.

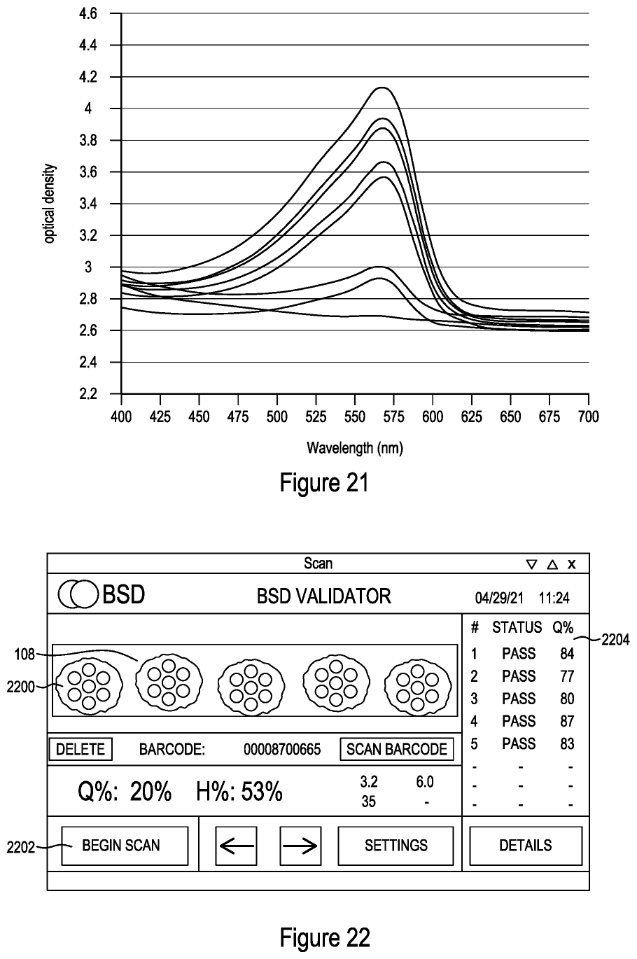

FIG. 21 depicts characteristic optical spectral density (light transmission) for saliva on pink indicating paper (Copan Nucleic-Card). This graph shows the best wavelength to achieve greatest contrast from white to pink dye areas on the sample card 204. When used for DNA card scanning, the LEDs ideally function at 565 nm.

FIG. 22 shows a user interface home screen on the display 106. The captured image 108 of the sample card filter paper area 204 is shown (example shows a 5 spot card). Computed punch sites 2200 (in this example 3.2mm diameter punches) are overlaid on the spots. In this example 35 punch sites of 3.2mm diameter were found. No 6.0mm punch sites were found. The displayed value Q % is an overall quality rating for the sample, based on numerous statistics. The displayed value H % is a hematocrit-based rating that indicates the consistency and range of the blood application. The values 3.2/6.0 are the pre-programmed punch sizes. The RH panel 2204 shows the spot number (left to right), the overall quality determination of each spot and the quality factor, computed from a range of statistics. The bottom buttons 2202 can be used to manually start an image scan, review previous/next sample result (history), open a Settings screen, and open a details screen.

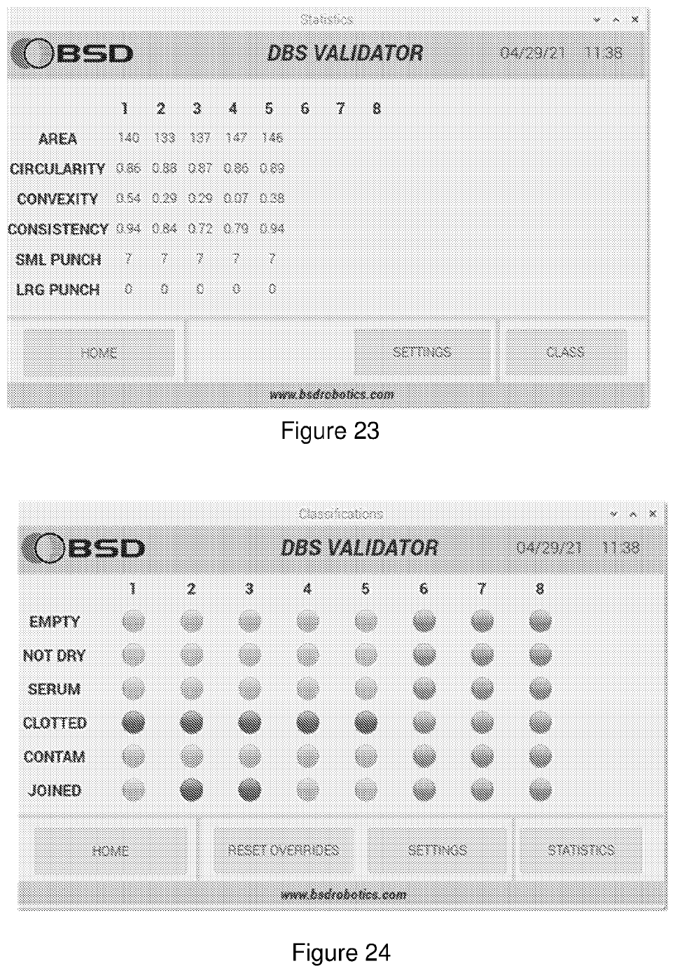

FIG. 23 shows the statistics screen of the user interface 106. The statistics screen shows results for each spot. The area is displayed in mm\hat{ }2, and other displayed circularity, convexity and consistency values are factors where 1.0 represent computationally perfect figure. The small punch values is the number found in spot (this case 3.2mm), whereas no large punches were found −6.0mm in this case.

FIG. 24 shows the classifications screen of the user interface 106. The class page shows the results from each of the classification algorithms. Green is a pass, Amber unsure, and red is a fail. Each button can be clicked to override by the operator. Overrides are saved in the record file for parsing to a machine learning algorithm to update the ML classification model.

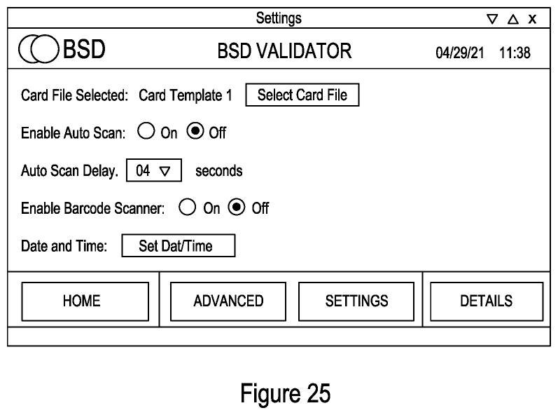

FIG. 25 shows the settings screen of the user interface 106. The user settings screen shows the basic settings that can be changed. Main parameters are contained in a card file that can be selected at the top. Select card file button opens a drop down to select. Auto scan will start the scan once the sample card 204 is detected in position. A delay can be set between card detection and card clamp activation/scanning for final position adjustment. The advanced button is used to open a command terminal window for maintenance and technician usage.

The embodiments show a method of passing controlled light through (transmissive) and against (reflectance) the sample and measuring geometric and light intensity features at one or more wavelengths.

FIGS. 5 and 11 show sample assessment apparatus 100, 100′, each having different physical formats. In one embodiment, the unit permits higher throughput in the form of a benchtop apparatus 100, the other is a more portable format that could be held in one hand. Both embodiments include a similar user interface to present results to the user.

Desktop Apparatus 100

Geometry and Enclosure

FIGS. 5 and 6 shows the overall geometry of the desktop apparatus 100. The desktop enclosure is arranged with a sloped front for the user interface 106 to allow comfortable working position either from a sitting or standing position.

The desktop apparatus 100 is required to process samples within a minimum of time and several features assist solve this need. Automatic sample presence detection 310 allows the instrument to automatically clamp 308 the sample card 304 and perform an image scan without user intervention. The detection of card presence can be performed by a separate photo-interrupter or reflectance-based sensor toward the rear of the sample loading area. This allows activation once the leading edge of the sample card 304 is sufficiently placed within the instrument.

Alternately the sample card 304 can be detected using the controlled light source 300 and image sensor 302 to continually monitor sample placement, with the light source 300 at minimum intensity and the image object edges detected at multiple points to determine the correct card insertion depth and alignment.

The appearance of the desktop enclosure is preferentially with a fine-textured surface finish, predominantly white with some coloured items, notably the sample entry area.

Card Guides, Platform and Clamp

FIG. 10 shows that adjustable card guides 1002 are provided to guide the edges of the card 204 into a consistent position and alignment for optical scanning.

The sample card 204 is partially inserted through an entry slot 1000 with a front ledge to an internal envelope, termed the card platform 707. The design of components in this area are rounded and designed as ‘lead-ins’ to guide the card easily into the instrument.

The card platform 707 comprises a frame that retains two sheets of optically transparent material, preferably glass 718, suspended above and below the sample card 204 so that light can pass vertically through the region of interest of the sample card 204. The properties of the transparent materials may include anti- reflective properties. Optionally the supporting glass may be laminated with material to filter light at a desired wavelength. Furthermore, the supporting glass may include antireflective coating and of a specific grade of glass to ensure optical clarity at the wavelengths of operation.

A clamping assembly 308 is activated prior to an image scan to ensure the card 204 is fixed in an acceptable location during imaging. A second function of the card clamp is to block light from entering through the slot 1000 which is the entry to a light tight chamber containing the controlled light source 300 and imaging system. The card clamp material could be a polyurethane coated bar 711 or coated metal that clamps the card down with some deformation against the card 204.

Optionally, LEDs incorporated on the outside surface of the entry slot 1000 further allows guidance and indication that the card clamp is active and holding the card 204 in place. One or more switchable light emitting diodes (LEDs) may be located at the card insertion port or entrance to enhance visual alignment of the sample card for the desktop embodiment. Once the sample is in place, the LED intensity could be modulated to indicate the scanning process and again that the card is ready to be removed.

Rear Stop

Given that card types may differ in size and geometry, the insertion depth into the apparatus 100 needs to match the card 204. An adjustable stop plate between the cover glass sheets allows the user to push the card in to a consistent depth prior to a scan.

The rear stop 708 could incorporate one or more miniature switch to detect the card 204 in place and is straight. The rear stop 708 would also be positioned so that the sample card 204 is clamped along an appropriate line on the card 204 away from the filter paper.

Further, the rear stop 708, if motorized, can be automatically set from to match the card geometry from a user setting. On completion of a scan, a motorised card stop 728 can assist to eject the card.

Light Box and Controlled Light Source

In the desktop apparatus 100, light from a 2-dimensional array of LEDs 739 is passed into a light box 715 of dimensions sufficient to minimise spatial variations of light exiting the light box 715. The light strikes a reflector surface of stable optical reflection in the visible range and is angled at 45 degrees to the incident light and to the upper glass support in the card platform 707.

The light box 715 therefore directs evenly dispersed light from the controlled light source 739 upon the top surface of the sample card 204. For purposes of illustration, the reflector surface is shown as planar in format and shape features may be incorporated to improve intensity distribution to correct for LED spatial brightness variations. An aperture device may be inserted between the reflector and upper glass support 718 to prevent stray light from exiting the instrument or to reduce unwanted internal reflections. Further, an area of light may be directed to a path outside the normal operating region of the sample card 204 to facilitate internal calibration of intensity and image exposure.

Mirror

The desktop apparatus housing incorporates an internal reflector, in the form of a mirror 719 to provide convenient geometry and sufficient optical path length from the image sensor 732 to the optical object (sample card 204). The mirror is mounted at 45° angle with respect to both the sample card 204 and the axis of the image sensor 732.

The image sensor 732 and a mirror 719 are used to capture the image from below the card 204, the relative positions of camera 732 and mirror 719 and size of the mirror 719 are determined so that the entry port is as close to the bottom of the instrument as possible.

Image Sensor and LEDs

The image sensor 732 is located in the rear of the housing 701 and attached to an internal assembly to minimise stray light and internal reflections, and in the shape of a rectangular cone having an internal surface finish to minimise light reflection.

The 2D image sensor 732 employed in the desktop apparatus 100 may be a monochrome or colour device. In the case of monochrome sensor, multiple images are taken with at differing LED colours to allow comparison of image at a plurality of wavelengths. It is preferred to use a colour image sensor to allow multiple wavelengths, substantially red and green, to illuminate the sample and use the colour channels of the image sensor to compare images at multiple wavelengths.

The 2D image sensor 732 includes an optical lens with focal length and image area able to capture the sample area of interest with acceptable spherical aberration and intensity homogeneity.

An area image sensor and appropriate data interface, implemented with a gate array or dedicated integrated circuit is required to allow data capture by the digital system. Suitable image sensors include items manufactured by On Semiconductor and Sony and include models such as AR1820HS, MT9J001 and MT9F002. Significantly, the image sensor 732 should have low noise characteristics and a wide dynamic range with 12-bit resolution to discern the low level of light experienced through dried blood spots in contrast to surrounding areas of white. The image sensor 732 is coupled with one or more lenses to focus light from the object 204 with minimal optical distortion.

Processor

The processor 304 for the desktop apparatus 100 is preferentially in the format of a single board computer with operating system to manage a range of peripherals and interfaces.

Controlled Light Source

Controlled light sources 300 are provided for front (reflective) and rear (transmissive) illumination of the sample card 204. In the case of the desktop apparatus 100, the rear light source 739 is comprised of multiple high intensity LEDs such as Cree Xlamp XQ-E LEDs arranged in a N×M rectangular grid. Groups of LEDs are controlled by dimmable current regulators, such as ST Microelectronics STCS1 on each series arrangement of LEDs to ensure consistent light emission.

The CPU 304 controls the intensity of all light sources 300 through pulse width modulation of the current regulators. Each LED device may be comprised of one or more colour LEDs to cover the required light wavelengths needed for discrimination of haemoglobin, notably at 565 nm and 620 nm. Optionally, a third wavelength at approximately 590 nm may be used which is a known isobestic wavelength for haemoglobin.

Portable Apparatus 100′

The portable apparatus 100′ requires attention to overall power consumption and compactness as key design criteria.

Geometry and Enclosure

The portable apparatus 100′ comprises a user interface 1203 incorporated into an ergonomic housing. The size and shape of the profile of the housings 1201, 1202 permit the user to perform sample presentation and operate the apparatus 100′ with ease by hand. The housing can be a single or multi-piece 1201, 1202. In the case of single piece, a sample holding mechanism, either electronically activated, or by hand, would secure the sample card 204 in place during optical scanning.

A two-piece design with a hinged arrangement of the portable embodiment is preferred, as this allows the apparatus 100′ to be opened for cleaning the interior of the instrument. One or more flexible cables or circuits are used to connect the electronic item, notably the display and touch-screen 1203, from the upper to lower housings, located in the hinge region of the enclosure. A door closed sensor for the portable apparatus 100′ prevents unwanted keypresses or activation when open.

In the portable apparatus 100′, the cover opens to allow the sample card 204 to be positioned, and when closed forms the light tight chamber and applies a force to retain the sample card 204 in a fixed position. When closing the cover, a locking mechanism acts to compress a spring and latch the cover against the lower body. Alternately, and preferentially, latches would be performed using permanent magnets located in the instrument housings 1201, 1202.

The display 1203 is recessed within the enclosure housing 1202 and incorporates a seal to prevent dust and moisture ingress.

Housings

The lower housing 1201 forms the base of the portable apparatus 100′ within which items including the main circuit board 1221, motor and drive mechanism 1208 for the contact image sensor, battery 1216, cover glass 1204 and hinge 1217 and latch components 1209 are mounted.

The lower housing 1201 incorporates features for the foregoing said items and includes a strip 1222 of deformable material with memory, such as polyurethane, that acts as a light seal and grip again the sample card 204 when the housing is in the closed position. The latching of top and bottom shells 1201, 1202 may be performed using a sliding mechanical arrangement and spring, or using opposing magnets 1209 embodied in the upper and lower housings 1201, 1202.

The upper housing 1202 includes the display 1203, illuminating backlight with controlled light source 1219, optionally, a sealed or tactile on/standby button 1218 and cover glass. The upper housing 1202 includes internal features for mounting and fastening of said items.

Image Sensor and LEDs

The contact image sensor 1207 is an assembly of linear CMOS detector array, a ‘self-focusing’ lens array (e.g. SELFOC®) and LEDs mounted adjacent to the lens array to illuminate the surface area. Reflected light builds charge within the detector array and produces a voltage that is sequentially sampled, pixel by pixel, as an analogue signal.

The image sensor 1207 for a portable apparatus 100′ comprises a contact image sensor 1207 that comprises a linear detector, linear lens array and arrangement of LEDs adjacent to the lens. Typical devices include M116-A6C1 P-A from CMOS Sensor Inc which provides an image over 108 mm wide and 2592 pixels. In this arrangement, the buffered video output is directly connected to the analogue input of the microcontroller 304 for fast conversion and assemblage of the image within internal or external RAM from each line scan.

The controlled light source 1219 comprises a rectangular LED illuminator or backlight panel sits over a sheet of cover glass 1204 to illuminate the card 204 from the top.

Processor

The portable apparatus 100′ is based on the use of a highly integrated mixed-signal (containing both analogue and digital capability) microcontroller (MCU) device 304 for high-speed analogue measurement functions with sufficient memory space to store complete images. An analogue interface is required to sample the video signal from the image sensor as shown in FIG. 17.

Optionally the portable apparatus 100′ could be coupled to a mobile device such as a smartphone, or a computer system, for example using wireless or USB interfaces.

In the case of the portable apparatus 100′, the digital system comprises components chosen for low power consumption with high performance such as the Microchip PIC32 series of 32-bit microprocessors.

The portable apparatus 100′ preferentially employs one or more single-chip microcontroller (MCU) 304 or digital signal processors (DSP) capable of low power operation and power management to run from a battery supply. Said MCU or DSP preferentially includes sufficient internal RAM memory for program and image data, and an internal analogue to digital converter (ADC) with sufficient sample rate to acquire image data from the contact image sensor with a suitable light response integration time. Alternately programmable logic devices may be used to implement part or all the digital processing functionality. In a preferential embodiment, a 32-bit low power MCU such as PIC32 MZ series includes sufficient memory and ADC with appropriate processing speed, peripherals, and power management to form the basis for an integrated system. In this embodiment the firmware runs without an operating system and using proprietary software.

Controlled Light Source

LEDs within the contact image sensor 1207 are used to illuminate the sample card 204 in a reflectance mode. The portable apparatus 100′ preferentially also employs a backlight system 1219, comprising material such as plexiglass and edge mounted LEDs driven by controlled light source components as illustrated in FIG. 12. The backlight 1219 is located close on the opposing surface of the upper glass retainer to pass light through the sample to the contact image sensor 1207. In this way the sample card 204 can be illuminated in transmissive mode, or if extinguished and the LEDs located within the contact image sensor 1207 enabled, in reflectance mode.

Motion Control

The contact image sensor 1207 is required to move across the sample area to acquire multiple line-scans to build a 2-dimensional image of the object. For this purpose, a stepper motor linear drive 1208 is used as an electronic positioner. The drive 1208 includes two H-bridge transistor drive outputs and a microswitch to recognize a zero position for the contact image sensor movement.

Power Management

DC power is supplied from an internal battery pack 1216 and circuitry 1220 to allow recharging from an external power adaptor is included. Internal voltage supply rails are regulated by low dropout voltage regulators. A micropower push button controller is used to put the device into low power standby and to provide a signal to wake the digital system from shutdown mode.

A standby controller in the power management portion of the system allows the system to automatically enter a low power state to extend the battery life of the system. In this regard, peripheral circuitry and substantially the processor shall be capable of micropower standby mode. Circuitry to allow peripherals without minimal power consumption is included to switch power off when in standby mode.

Common Aspects Between Desktop Apparatus 100 and Portable Apparatus 100 ′

Both apparatus' 100, 100 ′ include a graphic LCD to display the user interface 106.

Housings could be in the form of rigid plastic, such as acrylonitrile butadiene styrene (ABS), or other impact-resistant material. The surfaces of the housing may incorporate textures or features for decorative and branding purposes, for user comfort, or to increase the surface roughness for slip-resistance.

The texture and surfaces likely to receive greater user contact and particularly in the sample presentation area are arranged for ease of cleaning and minimal dust build-up. Rounded edges are used in areas subject to physical contact to ensure user comfort and safety, notably along the front area where the sample card is inserted.

Power Management

The enclosure may incorporate a separate battery compartment and may incorporate replaceable battery cells, for example “AAA” or “AA” or rechargeable or non-rechargeable types. Alternately one or more module rechargeable battery packs in the form of nickel/cadmium, nickel metal hydride, or lithium ion may be located within the housing.

Depending on system requirements, and substantially the processor and controlled light source, one or more voltage converters or regulators is included in the power management block to provide stable dc voltages, for example 1.8, 3.3,5.0 and 12V dc, with low noise that could affect image quality. One or more power converters and power switch or routing may be included for internal battery charging. Further, the power management portion of electronics may include one or more filtering or protection devices to limit conducted radiation, excess voltage or current.

The electronics may include power management circuitry for charging and a battery level monitor for indication to the user.

One or more power adapter ports provide connection of an external DC jack to provide convenient connection for system power, as an alternative to battery power, and may also simultaneously charge the internal batteries from an external ac-dc power adaptor.

One or more elements (for example multi-colour LED, and/or icon on the graphical interface) may be located on the housing to show the charging status and instrument on/off state.

Light Tight Design

The design of the seal 1222 that acts against the sample card 204 and the enclosure housings 1201, 1202 prevent light from entering the interior of the housing. Interior items, for example the light box 715 used for the controlled light source 740, provide secondary assurance of that internal areas are dark chambers until illuminated with controlled light sources.

The properties including material, colour and surface finishes of internal components are therefore chosen to either absorb or uniformly reflect the light, as required for each of the items. For those items that are required to absorb light or to minimise internal reflections, a black heavily texted or featured surface is used.

Controlled Light Source

The controlled light sources 300, 740, 1219 operate at high LED intensities and in order to produce stable light over time, temperature management may be required in the form of heatsinking and temperature monitoring.

Metallic heat sinks thermally bonded to the LEDs are used to reduce thermal resistance to air and dissipate heat over a greater air volume.

The inclusion of multiple controlled light sources 300, 740, 1219 allows illumination of the sample from either side and allows images to be captured in both reflective and transmissive mode. Imaging normally required from both sides to make sure sample has soaked through the card and not layered on the top.

Digital Electronics System

Both apparatus 100, 100 ′ include one or more printed circuit boards for the digital system, peripheral components, controlled light source components and associated power drivers.

Digital systems comprise a processor, non-volatile memory (for operating system, program and data storage) and random-access memory (RAM) of sufficient capacity for program operation and to contain image data.

Non-volatile memory can be in one or multiple forms including ROM, EEPROM or flash memory and removable storage devices such as secure digital (“SD”) memory card or USB memory device interfaces.

A real time clock is included to maintain current time and date updates and may be synchronised to or run independently of internet network time.

Communications

The system may include wired (“Ethernet”) or wireless (“Wifi”) connectivity and computer or an internet protocol (“IP”) network interface. Wired communication ports such as USB interfaces include devices to reduce the possibility of over voltage and conducted electromagnetic noise. One or more ports for connection of one or more wired communications, such as USB and for power are provided through openings in the rear or side of the housing.

Physical User Interface

The user interface 106 comprise only graphic LCD display with capacitive or resistive touch-screen interface, or optionally additional buttons, potentiometers, switches, LED indicators or other devices could be used to provide additional feedback and user controls during operation. Alternately, the apparatus 100, 100 ′ could be entirely or partially operated from a separate computer, using a suitable wired or wireless interface. It is noted that the use of a single touch screen display 106 reduces the number of penetrations through housing allows for easier sealing of the cases. This limits the potential for dust and fluid ingress which could affect internal components and contaminate surfaces in the path of light from source to image sensor 302, 302 ′.

The LCD display 106 includes backlighting to provide easy visualisation of the display and elements of the graphical interface are large to minimise eye strain and to allow fast operation of the touchscreen controls.

Barcode Scanner

Sample cards 204 may take a range of physical sizes and may or may not include a machine-readable identifier such as a printed barcode. Some form of identifier is preferred for the purpose of tracking and record generation. The apparatus 100, 100 ′ includes an optical barcode scanning device interface, to allow convenient reading of the sample card barcode, prior to, or as the card 204 is inserted to the apparatus 100, 100 ′. In an alternate embodiment, given sufficient image area, the barcode could alternately be read from the card image.

An internal or hand-held barcode scanner is optionally used to link the printed sample barcode with stored record files from scanning and stored sample images. Alternately the user may be presented with a virtual keypad to allow manual entry of a sample identifier.

Reports

The instrument is capable of exporting data records and associated images which are logged to non-volatile storage media. Such data export capabilities allow opening, manipulation and storage of the data using an external system or computer network. Optionally, the data can be stored in simple human readable or common formats such as text or JSON files or may be encrypted binary files for information security.

Output information from the instrument includes results of image analysis, date and time of scanning, barcode of sample card, and depiction of suitable punch locations in sample areas overlayed on an image of the sample card 204.

Results are compiled into record files and are stored within non-volatile memory. Results and images from previous scans can be accessed from the instrument interface using ‘forward’ and ‘back’ buttons on the user interface.

Calibration

Calibration of the apparatus 100, 100 ′ is required to account for dark count and image sensor response. The system embodies calibration, user settings and advanced configuration information. Said information can be set or changed using the incorporated user interface or preferentially via a communications port to an external computer system with greater capacity to display and present settings, parameters and calibration information in graphical format, including graphs and images.

Graphical User Interface

Each screen shown in FIGS. 22 to 25 has common features:

Header Bar at the top of the screen with branding information and current date and time.

Virtual button arranged along the bottom of the display.

The visual appearance of buttons changes to indicate which is the current screen.

A status message window is used to display processing steps

Colours indicate:
a. Green—pass
b. Amber—questionable
c. Red—fail

The graphical interface and displays could be performed by an embedded processor or by an external computer system. The graphical user interface is presented on a display 106 which may have a touch sensitive means for user interaction.

The screens for the portable apparatus 100 ′ are substantially equivalent as set out below, however due to a more compact screen size (7″ for the desktop embodiment and 5″ for the portable) and user interface 106 is arranged in alternate and more compact format for the portable apparatus 100′.

The user interface 106 includes user accounts and a login page to ensure that username and permissions are managed by an administrator. Username is recorded in the record file to allow tracking of results to a specific collection event. Optionally the portable apparatus 100′ may include a global positioning system (GPS) receiver peripheral to record site location.

Home Screen

On bootup, the Home screen is the default and the Scan button is highlighted when ready for operation. The scan button is like a home function and returns to the Scan screen ready to take the next sample. Pressing the scan button will remove the previously scanned information ready to process the next card 204.

The home screen provides controls and displays results for each sample scan. It includes virtual buttons to allow navigation to various pages as shown in the figures.

Virtual ‘arrow’ buttons allow a user to scroll through previous scans and show results and images in a fashion similar to the image review function for a digital camera.

Details—Classifications Screen

The user interface 106 provides both summary and detailed information for a range of user types.

Conditions are determined from rule-based comparisons and optionally from a machine learning classifier algorithm, including:

Insufficient sample, where the area of acceptable sample falls below a value that is set as a parameter.

Scratched or abraded surface, detected from optical inconsistences across the sample area.

Sample not dried before packaging, is detected from wavelength comparison of image pixels within the sample area and in the case of indicating paper, the contrast between sample and bounding areas of the collector.

Supersaturated or clotted sample—where excess of biofluid has been applied to one or both sides of the collector, based on image intensity of the sample area.

Sample contamination, dilution or discoloration, based on wavelength discrimination and consistency across the sample area.

The classes screen shows result of sample classification in easy to recognize visual indication in the form of coloured buttons. Clicking on a button causes the button to change colour and are saved as ‘overrides’ for the purpose of data collation for machine learning. The buttons are green to indicate a passing result, amber for unsure and red for fail. The user simply touches the user selectable buttons to override the classification.

Details—Statistics Screen

The statics results screen breaks down results per sample spot, of which the number of spots is obtained from a selectable card file associated with specific sample card types.

Advanced user settings including values of thresholds, punch sizes, card formats are stored within the card file.

In general, green is used to show a passing result, amber to indicate a marginal result and red to denote a failed result.

Settings Screen

Selection of options for user controls is limited to basic operations and more advanced details of image processing and classification are embodied in a card file. This allows processing of various types and formats of sample cards, or for different conditions by selecting the appropriate card file.

If the AutoScan option is not selected in the Settings then the Scan button must be pressed before scanning a new card 204. With AutoScan enabled then once the card 204 has been detected then the software will automatically scan the card and update the Scan information, comprising the image and variables.

The user can change the date and time, clicking a button brings up a date time editor.

Operation

If barcoding is enabled:

The status bar prompts to “SCAN BARCODE” and operation pauses until a barcode has been received. The barcode is saved and used to create the internal record file. The barcode ID number is shown on the screen. If AutoScan mode is enabled the status bar prompts “INSERT CARD” and operation waits until a card 204 is detected then starts a scan. If AutoScan is off then the software waits for the card 204 detection AND a press of the Scan button before proceeding with a scan.

If barcoding is disabled

A unique ID is created by the software based on date and time and is used to create the internal record file and is shown as the ID number on the screen. If AutoScan mode is enabled the status bar prompts “INSERT CARD” and operation waits until a card 204 is detected then starts an image scan. If AutoScan is off then the software waits for the card detection AND a press of the Scan button before proceeding with a scan.

A person skilled in the art will appreciate that many embodiments and variations can be made without departing from the ambit of the present invention.

The apparatus may be configured to process the samples from a stack so that individual sample presentation is not required, thereby minimizing human handling and time.

The apparatus may include an integral printer, or printer connection, for traceability information printing such as processing date and time, and identification information for patient and/or collection site. Similarly, a concise report of sample quality can be printed on the sample card, along with any instructions for processing in the central laboratory and for subsequent storage tracking.

The apparatus can be interfaced with an internal or external scanning device to capture images of hand-written patient demographics, with optical character recognition for automated patient record creation.

The apparatus may include means for geographical map location recording.

The sample card may comprise filter paper impregnated with an indicating dye that changes colour when a liquid such as saliva is applied. In this context the apparatus determines sample quality based on statistical analysis of colour change intensity and area on the sample card. Computation of statistics may be used to determine the optimal locations for removing subsamples containing, for example, sufficient saliva sample for forensic DNA sample analysis.

In compliance with the statute, the invention has been described in language more or less specific to structural or methodical features. It is to be understood that the invention is not limited to specific features shown or described since the means herein described comprises preferred forms of putting the invention into effect.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

1. A biological sample quality apparatus for determining the pre-analytical quality of a biological sample, the biological sample including dried biofluids on filter paper, the biological sample quality apparatus comprising:
a sample receiver configured for receiving the biological sample;
one or more light sources configured for supplying light to the biological sample, the one or more light sources including a reflector for reflecting light from within a lightbox that directs controlled light toward the biological sample to provide acceptable image quality free from unwanted reflections and spatial light variation;
an image sensor configured for capturing an image of the lit biological sample; and
an image processor configured for image processing the captured image to determine the quality of the biological sample.

2. The biological sample quality apparatus as claimed in claim 1, wherein the sample receiver includes a dock for docking a sample card, directly or indirectly, bearing the biological sample; and/or the dock including a guide or slot so that a sample card can be inserted into the biological sample quality apparatus in a consistent and repeatable manner.

3. The biological sample quality apparatus as claimed in claim 1, wherein the sample receiver includes a clamping mechanism to temporarily clamp the biological sample in position, and/or a detector to detect for the presence of a sample card.

4. The biological sample quality apparatus as claimed in claim 1, wherein the sample receiver includes a moving mechanism to automatically move the biological sample to and from an image capture position.

5. The biological sample quality apparatus as claimed in claim 1, wherein the light sources provide one or more specific wavelengths, and/or can be adjustable for intensity and/or exposure time.

6. The biological sample quality apparatus as claimed in claim 1, wherein the one or more light sources include a transilluminator whereby the light emanates from a planar surface.

7. The biological sample quality apparatus as claimed in claim 1, wherein the one or more light sources include a light-safe enclosure with minimal internal reflections to ensure integrity and homogeneity of the captured image, the light sources including one or more colored LEDs to acquire images at multiple wavelengths.

8. The biological sample quality apparatus as claimed in claim 1, further comprising an identifier for identifying the biological sample, the identifier reading a printed barcode or radio frequency identification tag.

9. The biological sample quality apparatus as claimed in claim 1, wherein the image sensor utilizes reflective and/or transmissive scanning of the biological sample, the image sensor being a one dimensional (1D) or two dimensional (2D) scanner.

10. The biological sample quality apparatus as claimed in claim 1, further comprising:
a controller configured for controlling the light source and image sensor to capture digital images, the controller including the image processor, and processing and storing captured images, performing numerical computations, and/or interfacing with network and data storage devices;
an exterior housing configured to facilitate portability and incorporating a touch screen interface;
an internal battery supply configured for supplying the apparatus in areas where power connection is not available;
internal or external storage media configured for storing captured images; and/or
optical filtering configured to allow specific wavelengths of light to be clearly discerned;
and/or
a communications port configured for integration to a Laboratory Information System (LIS) or other data system over computer network connections.

11. A biological sample quality method for determining pre-analytical quality of a biological sample, the biological sample including dried biofluids on filter paper, the biological sample quality method comprising:
receiving the biological sample;
supplying light to the sample via a reflector which reflects light from within a lightbox that directs controlled light toward the biological sample to provide acceptable image quality free from unwanted reflections and spatial light variation;
capturing an image of the illuminated sample; and
image processing the captured image to determine the quality of the sample.

12. The biological sample quality method as claimed in claim 11, further comprising spatial computation for downstream removal of one or more acceptable areas of an acceptable sample for subsequent analysis to provide accurate and repeatable results.

13. The biological sample quality method as claimed in claim 11, wherein receiving the biological sample includes detecting a presence of the biological sample and clamping the biological sample in a fixed position.

14. The biological sample quality method as claimed in claim 11, further comprising identifying the biological sample whether by activating a barcode scanner to automatically record a sample identifier or digitally processing the identifier from the captured image.

15. The biological sample quality method as claimed in claim 11, further comprising compensating the captured image prior to display to an operator and/or storage, the compensating involving spatial and/or intensity correction.

16. The biological sample quality method as claimed in claim 11, wherein capturing includes optical filtering to allow specific wavelengths of light to be clearly discerned.

17. The biological sample quality method as claimed in claim 11, wherein the image processing includes:
statistical or logistic processing of one or more captured images, from one or more viewing positions of the biological sample, and at wavelengths of the supplied light to determine the quality of the biological sample;
determining an acceptable dried sample area including location, area and spatial arrangement including boundaries;
assessment of sample density and consistency across the biological sample;
classifying the biological sample from statistical data, the statistical data relating to blood hematocrit and age of sample estimates based on wavelength comparisons;
computation of human-relatable metrics allowing direct comparison of human visualization with machine image processing results;
a final pass or fail result; and/or
determining an overall quality factor by assessing image processing values against parametric constants which are able to be adjusted by an operator.

18. The biological sample quality method as claimed in claim 11, wherein the image processing includes computation of acceptable sampling regions of the sample, the regions being determined using parameters relating to the circularity of sample spot; consistency of image values within each sample spot; texture analysis of the sample spot; image area comparisons based on wavelengths or ratios of wavelengths; or from user-specified values relating to specific laboratory workflow, such as the number and size of sub-samples to be removed from the sample.

19. The biological sample quality method as claimed in claim 11, wherein the image processing includes determining the centroid coordinates of suitable circular, of a specified diameter, areas containing sample areas to be removed, the centroid coordinates being referenced to a datum point on a sample card so that the card can be inserted to other equipment for automatic removal of the areas to be removed.

20. The biological sample quality method as claimed in claim 11, further comprising:
storing a data record including the captured image, computed information including statistics and/or sample identification details; and/or
computing, displaying and storing classifications relating to the sample, the user preferably having the facility to override classifications if the user disagrees with a displayed classification, allowing subsequent improvement of classification algorithms based on user feedback.
